(19) United States
(12) Patent Application Publication
(10) Pub. No.: US 2015/0342190 A1
(43) Pub. Date: Dec. 3, 2015

(54) METHODS FOR LEVERAGING HORMESIS IN PLANT BREEDING AND PLANTS WITH ENHANCED HORMESIS EFFECTS

(71) Applicants: PIONEER HI BRED INTERNATIONAL INC, Johnston, IA (US); E I DU PONT DE NEMOURS AND COMPANY, Wilmington, DE (US)

(72) Inventors: KEVIN MCGREGOR, Ankeny, IA (US); Scott Anthony Sebastian, Polk City, IA (US); Stephen Douglas Strachan, Oxford, PA (US); Mark D. Vogt, Ankeny, IA (US)

(21) Appl. No.: 14/722,348

(22) Filed: May 27, 2015

Related U.S. Application Data
(60) Provisional application No. 62/005,380, filed on May 30, 2014.

(51) Int. Cl. A01N 47/36; C12N 15/01; A01N 57/20

(52) U.S. Cl. CPC A01N 47/36; A01N 57/20; C12N 15/01

(57) ABSTRACT

Methods for plant breeding using hormesis effects as selection criteria are disclosed. Plants enhanced with strong hormesis responses can be obtained with the methods. Improved seedling vigor and improved yield by application of herbicide to herbicide tolerant plants is demonstrated. Improved cold germination in herbicide tolerant plants is demonstrated.

### CROSS-REFERENCE TO RELATED APPLICATIONS

This application claims priority under 35 U.S.C. §119 to provisional application Ser. No. 62/005,380 filed May 30, 2014, herein incorporated by reference in its entirety.

### BACKGROUND

Hormesis is defined broadly as any positive biological response to sub-lethal concentrations of a substance that is toxic or lethal at higher concentrations. It is known that certain plants have favorable growth characteristics when exposed to low doses of herbicide that are fatal to the plants when administered in higher doses. It is also known that certain characteristics including protein content, resistance to pathogens, plant weight, and height can be enhanced under certain circumstances by applying low doses of herbicide. However, positive hormesis responses are notoriously unpredictable/unreliable and therefore difficult to harness for commercial purposes.

While hormesis as a natural phenomenon has been known, the agricultural industry has not enhanced plants by breeding and/or specifically selecting for plants with enhanced hormesis responses trait(s). While there is an ever-present need in agriculture for more vigorous plants with enhanced favorable characteristics including seedling vigor, biomass production, seed yield, oil content, protein content, disease resistance, pest resistance, cold tolerance, drought tolerance, and nutrient deficiency tolerance for example, no methods existed before this invention of breeding and/or selection for plants with an enhanced hormesis response.

### SUMMARY

It is an object of the invention to provide methods of leveraging hormesis in plant breeding and plants with improved and/or more consistent hormesis response through plant breeding.

### BRIEF DESCRIPTIONS OF THE DRAWINGS

FIG. 1. Graphical illustration of hormesis and hormesis enhancement using an herbicide tolerance trait.

FIGS. 2a-d. Hydroponic system modified for the current invention. Nutrient solution supplemented with 0.35 PPM rimsulfuron and 22 PPM glyphosate inhibited growth of all plants except those homozygous for Als1+Als2+RR. FIG. 2a: Commercially available HydroFarm® MegaGarden system. FIG. 2b: Modified system with smaller pots and selective herbicides. FIG. 2c: Response of known genotypes at HR loci. FIG. 2d: Variation among plants fixed for Als1+Als2+RR.

FIG. 3. Range of visual scores observed among plants of synthetic bulk population.

FIG. 4. Phenotypic (visual score) and genotypic (MAS score) distribution among plants of synthetic bulk.

FIG. 5. Graphical depiction of yield improvements observed for certain herbicide tolerant genotypes upon herbicide application.

FIG. 6. Graphical depiction of cold germination improvements observed for certain herbicide tolerant genotypes.

FIG. 7. Percent germination over time of soybean lines exposed to two different temperature regimes.

### DETAILED DESCRIPTION

Hormesis has been proven in both plant and animal species and is even leveraged for commercial purposes in fields such as human medicine. However, hormesis is unknown as a useful tool in the field of plant breeding, perhaps because at the low doses needed to provide beneficial effects any herbicide would be ineffective for pest control in commercial crop production. Hormesis effects are notoriously difficult to predict and control and hormesis has previously only been documented at herbicide concentrations that are well below the range needed for commercially-effective weed control. But now that many crop plants are bred to contain major-effect genes for resistance to one or more herbicides, positive responses to these herbicides may become more common at concentrations that are effective for weed control or residual from previous crop rotations.

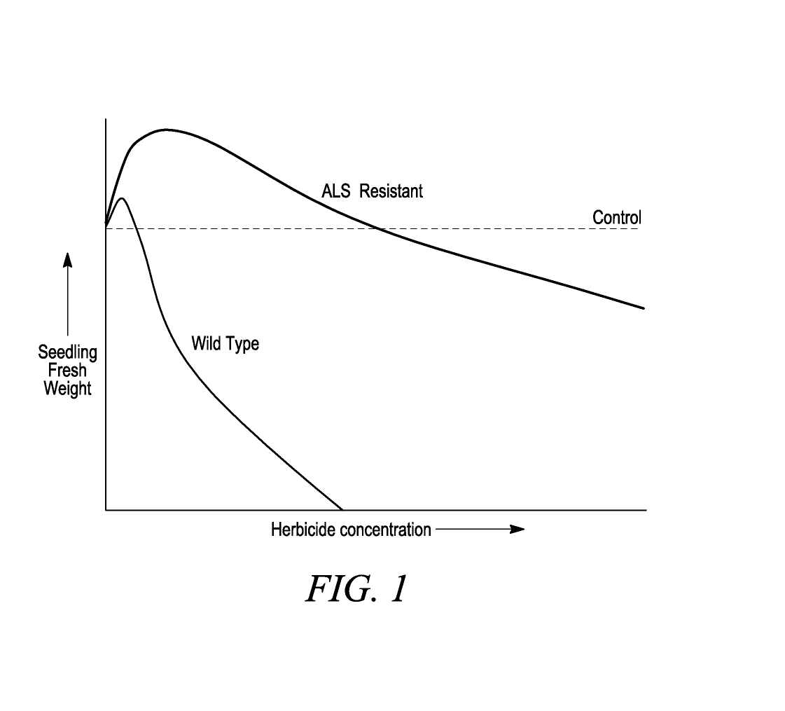

FIG. 1 provides an illustration of hormesis and how it can be enhanced using the methods of the invention. A measure of vigor, in this example seedling fresh weight, is plotted on the y-axis against application of increasing amounts of a stress, in this case herbicide. Even wild-type plants that have no resistance to the herbicide show an increased seedling fresh weight upon application of small amounts of herbicide as shown by the area of the wild type curve that exceeds the control (no herbicide application) seedling fresh weight. This improved vigor upon application of small amounts of herbicide is a hormesis effect. When the plant is herbicide tolerant, however, an increased maximum extent of hormesis (improved seedling fresh weight) is observed along with an increased herbicide concentration that can be applied to achieve the maximum hormesis effect, i.e., the peak seedling fresh weight is achieved with a greater herbicide concentration. This has the dual benefit of increasing the hormesis effect along with improved tolerance to herbicide allowing the herbicide to be applied at concentrations effective for weed control.

Hence, through the methods of the invention, hormesis can be an important response to leverage in plant breeding and commercial plant production. Previous methods to select for single or multiple toxin resistance genes are laborious, expensive, and/or imprecise. In addition, previous methods do not recognize, select for, or have the precision required to efficiently identify and select plants with maximum hormesis response. The invention thus includes plant breeding methods that select for plants with greater hormesis response than other plants.

The present invention includes methods to quickly identify rare plants that contain known genes for toxin resistance in addition to unknown genes that promote hormesis. These methods dramatically improve the efficiency of plant breeding—especially for crops that are routinely exposed to one or more herbicides and/or other toxins during commercial production. A feature of this invention is that it does not require prior knowledge of hormesis-related genes. Instead, selection can be based on a precise whole plant assay that quickly identifies plants containing rare combinations of genes that reduce the negative while promoting the positive effects of herbicides or other toxins or stressors.

This invention provides novel plant breeding methods that maximize the potential for hormesis to increase crop yields—especially with herbicide application rates needed for effective weed control under field conditions. The invention also provides methods to quickly screen large segregating breeding populations for rare plants that contain single or multiple known HR genes in addition to known or unknown genes that improve herbicide efficacy and/or maximize the probability for hormesis under commercial crop production conditions. This invention can also be modified to identify plants that respond favorably to any toxin or combination of toxins including herbicides, insecticides, fungicides, plant growth regulators, and/or environmental toxins/stressors.

The invention includes a hydroponic system to permit rapid planting and uniform growth of densely-planted seedlings. The hydroponic system may be irrigated with an aqueous solution containing a sufficient concentration of one or more toxins such that plants containing a gene or genes that confer resistance to the toxins can be easily identified by their differential ability to grow in the presence of the toxins. The toxins can be added to the aqueous irrigation solution simultaneously or sequentially depending on which method provides the best results. This system provides optimal environmental conditions for subsequent growth such that further selection for vigor and/or biomass accumulation among the toxin resistant plants can be used to identify plants with known and/or unknown genes that improve resistance and/or promote hormesis in the presence of the toxins. Using the methods of the invention, plants may be selected and subsequently grown to maturity in the same hydroponic culture system, or they may be transplanted to soil to produce multiple progeny for subsequent testing. Biomass or seed yield differences among selected plants can then be used to identify plants with maximum vigor for subsequent trials. In one embodiment, marker assisted selection (MAS) may be used to confirm the zygosity of a genetic trait after selection, for example, an herbicide resistance gene. Hydroponic selection can also dramatically improve plant breeding efficiency by focusing MAS resources on the few plants that survive the hydroponic screen. In this case, MAS of only the selected plants (as opposed to the entire population of plants) would be used to confirm that the selected plants are indeed homozygous for known HR genes. In another embodiment, the selected plants can be genotyped with whole genome markers to generate a quantitative whole genome prediction of fresh weight accumulation and/or seed yield. This can further improve the heritability of selection and can identify the genomic location of previously unknown genes that improve plant health and/or yield in the presence of toxins.

Many types of whole plant assays could be used to screen for known and unknown herbicide response genes, but hydroponic culture systems are preferred for precision, throughput, and repeatability of results. Hydroponic culture systems vary greatly in size and purpose depending on the precision and throughput needed. For example, in one greenhouse or growth chamber, hydroponic systems are easily scalable to permit screening even millions of plants in 7 to 14 days. Once the healthiest-looking plants are identified, they can be transplanted into herbicide-free pots and grown to full maturity to produce progeny seed. Multiple progeny from each of the selected plants can then be used to confirm the genetic purity of each selected plant, to fix genetic loci of interest in the homozygous condition, and/or to confirm the herbicide response phenotype of selected progeny under a wide variety of greenhouse, growth chamber, and field conditions.

Pilot studies (7 to 14 days each) can be used to determine which concentrations of each herbicide(s) can be added to the hydroponic irrigation solution to visually differentiate between control plants of known HR genotype and purity. Many types and sizes of inexpensive hydroponic systems are commercially available such that multiple hydroponic units with different treatments can be run simultaneously in the same greenhouse, growth chamber, or field.

The invention includes seed production techniques in which the seeds are enhanced with improved properties by growing the parent plant under stress conditions. For example, seeds may be produced by plants grown under water restricted conditions, in the presence of one or more pests, or under the stress of toxins. We have found unexpectedly that seeds produced by parent plants grown under stress have improved qualities including drought resistance, pest or toxin resistance, and improved vigor.

### DEFINITIONS

“Hormesis”: any positive biological response to sub-lethal concentrations of a substance (or environmental stress) that is toxic or lethal at higher concentrations.

“Herbicide resistance” or “herbicide tolerance”: a plant trait that is observable as the ability of said plant to develop normally or display minimal damage when exposed to one or more herbicide treatments that severely inhibit development or kill other plants of the same species.

“Herbicide resistance gene” or “HR gene”: Any gene that has been previously characterized and determined to confer resistance to one or more herbicides when present in most or all genetic backgrounds of a given plant species. Examples of commercially relevant HR genes used in soybean breeding and production include but are not limited to Als1 (sulfonylurea resistance), Als2 (sulfonylurea resistance), Roundup Ready® aka RR™ or RR (glyphosate resistance), Roundup Ready 2 Yield™ aka RR2 or RR2Y® (glyphosate resistance), LibertyLink® aka LL (glufosinate resistance), Enlist™ (2,4 D resistance), Xtend®™ (dicamba resistance), Optimum® GAT® (glyphosate resistance), Hm (metribuzin resistance). This list is not exhaustive and other herbicide resistance genes will be known to those of ordinary skill in the art.

“Modifier” or “modifier gene”: Any known or unknown gene or quantitative trait locus (QTL) that enhances the expression of one or more major-effect genes (such as an HR gene). Modifier genes may or may not have an effect in the absence of HR gene(s) and may only be expressed in certain genetic backgrounds. Evidence for modifier genes can be implied by their differentiating effect on herbicide efficacy in some, most, or all genetic backgrounds and/or by their association with molecular markers that cosegregate with an herbicide resistance phenotype in some, most, or all genetic backgrounds.

“Epigenetic modifier” or “epigene”: A modifier gene that is not normally transcribed (due to methylation or other reasons) but may be reactivated in response to some type of environmental stress. For example, epigenetic modifiers of HR genes may become activated when plants are exposed to herbicide stress and/or other stresses that may or may not be obviously related to herbicide response. These activated gene(s) may also be heritable in subsequent generations of progeny—especially when each generation of plants is exposed to the same environmental stress.

“Efficacy”: The relative level of herbicide resistance conferred by a given HR gene, combination of HR genes, or combination of HR and modifier gene(s). Efficacy is usually determined by increasing the concentration and/or number of herbicide treatment(s) until measurable differences are observed among herbicide resistant plants. For example, the combination of Als1+Als2 HR genes confers much higher efficacy to ALS-inhibiting herbicides than either Als1 alone or Als2 alone. Herbicide efficacy can also be increased by combining known HR gene(s) with known and/or unknown modifier gene(s).

“MAS” or “marker assisted selection”: Selection of plants based on a molecular assay of the gene(s) conferring a given trait/phenotype. A desirable feature of MAS is the ability to directly determine genotype without the need to expose plants to the precise environmental conditions required to observe the desirable trait in a whole-plant assay. Possible undesirable features of MAS include the infrastructure and cost versus other assays and/or the a priori need to know the causal genes or genetic markers linked to the desired trait gene(s).

### EXAMPLES

### Example 1

This example demonstrates the use of a fast, efficient, and precise whole plant assay to identify plants that contain known major-effect herbicide resistance (HR) genes in addition to other known or unknown genes that enhance the efficacy of HR genes and maximize the potential for positive hormesis responses.

Pilot studies were conducted over several months to determine that continuous exposure to a solution containing 0.35 PPM rimsulfuron and 22 PPM glyphosate severely inhibited emergence and/or subsequent growth of all plants except those that were homozygous for three HR genes Als1+Als2+RR where RR=either RR® or RR2Y®. Once the selective herbicide rates were determined, hundreds of seeds from populations segregating for Als1, Als2, and RR were quickly screened to identify and select the small subset of plants that were homozygous resistant at all 3 HR loci. After 7 to 14 days in the hydroponic system, the healthiest looking plants were visually identified and transplanted into the greenhouse, growth chamber, or field to permit maximum growth, maturation, and seed increase for subsequent testing under many different environmental conditions. During the transplanting process, subtle quantitative differences among the selected plants were rated visually and were also measured with weigh scales. Spectral devices and other instruments may also be used to detect differences among plants, although in this example only visual ratings and seedling weight were used. These more subtle differences were used to detect the presence of previously-unknown genes that maximize herbicide resistance and/or hormesis response in combination with major HR genes. The rescued plants or progeny were genotyped with molecular markers to confirm genetic purity at HR loci and to identify previously-unknown genes that further enhance efficacy and/or hormesis.

The efficiency of a hydroponic assay for both known and unknown herbicide response genes is demonstrated in this example. In this case, 4 different soybean lines of varying genotype at the Als1, Als2, and RR loci were used to demonstrate rapid and effective visual selection of lines homozygous for Als1+Als2+RR. Evidence of other loci that modify herbicide response is also demonstrated by variation in both visual phenotype and fresh weight accumulation of plants confirmed to be homozygous for Als1+Als2+RR by genetic markers.

### Materials and Methods

### A. Soybean Lines of Known Genotype at 3 Major Effect HR Loci

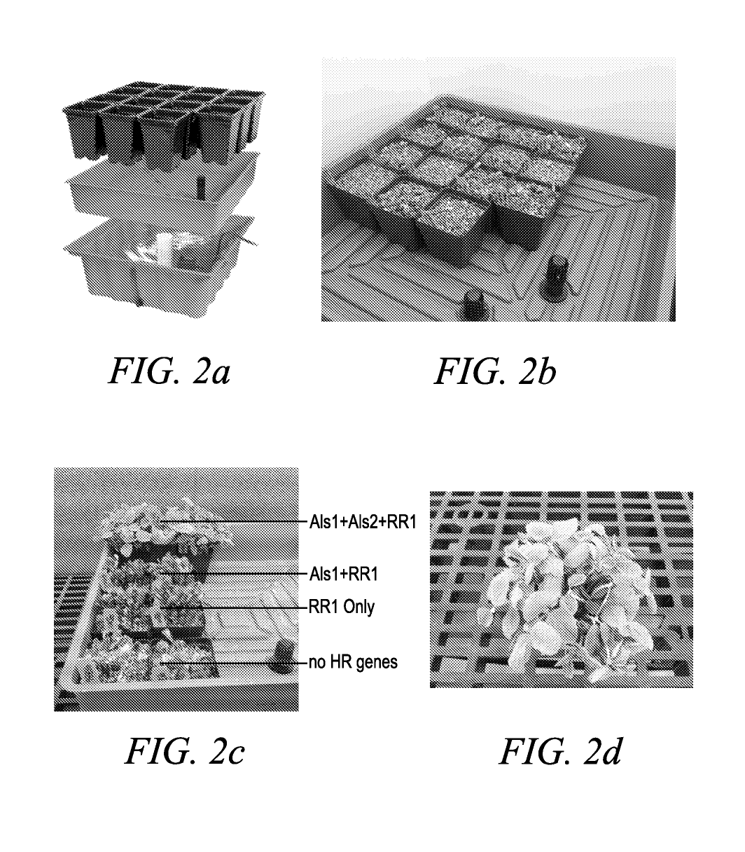

Four different soybean lines (Table 1) of known genotype at the Als1, Als2, and RR loci were used to test the effectiveness of a whole plant hydroponic assay to identify plants that are ‘fixed’ (homozygous and homogeneous) for all three HR genes Als1+Als2+RR. In pilot studies, plants of these lines grown in separate pots (as shown below in FIG. 2c) indicated that a combination of 0.35 PPM rimsulfuron and 22 PPM glyphosate effectively inhibits emergence and/or development of true leaves beyond the point of cotyledon expansion of any plants from the lines in Table 1 that are not homozygous for all three HR genes. These herbicide concentrations are also high enough to cause visual but non-lethal injury of plants fixed for all 3 HR genes (FIG. 2d). This was done intentionally to insure stringent selection for all 3 genes (i.e. no false positives) and also to look for evidence of previously-unknown genes that modify herbicide response even when all 3 major HR genes are known to be fixed.

TABLE 1
Soybean lines used to create synthetic bulk population for hydroponic assay

| | | | # of seeds bulked |
|---|---|---|---|
| | Fixed at HR | Relative purity at | to mimic a |
| | loci for these | other genome wide | segregating |
| Line Name | genes | loci | population |
| 93B86 | none | fixed | 250 |
| 93M11 | RR | fixed | 250 |
| XB41T13 | Als1 + RR | fixed | 250 |
| BC44883270 | Als1 + Als2 + RR | segregating | 250 |
| synthetic | bulk of above | segregating | 1000 |
| population | lines | | |

According to pilot studies, plants of BC44883270 should be the only plants listed in Table 1 to survive a combination of 0.35 PPM rimsulfuron and 22 PPM glyphosate in hydroponic culture; i.e. this is the only line in Table 1 that is fixed for Als1+Als2+RR.

### B. Hydroponic Screening System

Many types and sizes of inexpensive hydroponic systems are commercially available such that multiple systems with different treatments can be run simultaneously under uniform growth chamber or greenhouse conditions. These systems are designed to be irrigated with nutrient solutions that maximize plant health and growth. For selection purposes, the nutrient solution can be supplemented with herbicide(s) at sufficiently-high concentrations to inhibit growth of plants that do not contain major HR genes. Herbicide concentrations can also be adjusted to stress plants that are fixed for known HR genes such as Als1+Als2+RR or any other combination of known HR genes. When plants with known HR genes are stressed, visual differences among plants can be used to select for other genes (known or unknown) that improve efficacy of the HR genes and/or maximize the hormesis response. For small pilot studies and screening purposes, multiple units of the HydroFarm® MegaGarden hydroponic system were used (available at hydrofarm.com). This is just one example of a commercially-available whole plant screening system that could be used directly or modified to screen for herbicide and/or other toxin response genes. Similar hydroponic systems are also commercially-available, inexpensive, and easily scalable to screen millions of plants if necessary.

Each hydroponic system (FIG. 2a) consists of a tray (56 cm×56 cm×12 cm) that sits on top of a 28 liter reservoir tank (56 cm×56 cm×20 cm). The top tray can be modified for use as one large planting pot or used to hold multiple smaller pots with drainage holes as shown in FIG. 2b. The system comes with its own planting pots (FIG. 2a) but these were replaced with smaller and shorter pots (8.9×8.9×8.9 cm) such that each system can hold a maximum of 36 small pots (FIG. 2b). Each small pot can be used for planting of multiple seeds of a known genotype (e.g. as a control) or to plant multiple seeds of a population that is segregating for known and/or unknown herbicide response genes.

Continuous and uniform exposure of seeds/seedlings to the nutrient+herbicide solution was enforced by a programmable timer controlling a pump inside the nutrient solution reservoir. In this example, the pump was programmed to flood the upper planting chamber for 15 minutes once every 8 hours. The depth of flooding in the upper chamber was controlled by an adjustable overflow drain that maintains a uniform depth of the nutrient/herbicide treatment throughout the planting medium that contains the seeds or developing seedlings. The irrigation solution drained back into the bottom reservoir after the desired flooding period (around 15 minutes) that occurs at the time interval desired (here, 8 hours).

To promote uniformity of treatment in the upper planting/growth tray, each pot was filled with an inert planting medium (course vermiculite) that drained well while retaining enough moisture to prevent desiccation between irrigation cycles. Trays, water pump, irrigation tubing, and an electronic timer were included with the commercially available hydroponic system. The nutrient reservoir tank of each hydroponic system was also supplemented with an ‘air stone’ connected to a small aquarium air pump to insure that the nutrient solution was well oxygenated. This was an added precaution to promote uniformity and optimum growth conditions.

### C. Herbicides and Herbicide Stock Solutions for Use in Hydroponic Culture

Pilot studies indicated that a combination of 0.35 PPM rimsulfuron and 22 PPM glyphosate effectively inhibits emergence and/or development of true leaves beyond the point of cotyledon expansion of any plants that are were not fixed for all three HR genes Als1+Als2+RR. Rimsulfuron is an ALS inhibitor of the sulfonylurea (SU) class and the active ingredient (ai) in Resolve®SG herbicide. A 10,000 PPM concentrated stock solution of rimsulfuron was made by adding 40 g of Resolve®SG (10 g ai) to 1000 ml of RO water. 0.035 ml of stock solution was then added per liter of nutrient solution in the reservoir tank to achieve a final concentration of 0.35 PPM rimsulfuron in the hydroponic nutrient solution. Glyphosate is an EPSP synthase inhibitor and the active ingredient in Roundup PowerMAX® herbicide. A concentrated stock solution of 35,000 PPM glyphosate was made by adding 65 ml of Roundup POWERMAX® to 1000 ml of water. 0.64 ml of the stock solution was added per liter of nutrient solution in the reservoir to achieve a final concentration of 22 PPM glyphosate in the hydroponic system. Water level in the nutrient reservoir (described below) was monitored and supplemented with water to replace water lost through evapotranspiration that occurred over the course of the experiment.

### D. Hydroponic Assay, Visual Scoring, and Confirmation Via MAS

After establishing the proper herbicide concentrations for selection of plants fixed for Als1+Als2+RR, 250 seeds of each of the 4 lines listed in Table 1 were mixed together to create a ‘synthetic bulk’ population of 1000 seeds that could be screened en masse (as opposed to keeping each line in separate pots). Mixing of seed was done to more closely mimic the competition among densely-planted seeds of varying genotype that breeders would experience when screening breeding populations that segregate for all 3 HR genes.

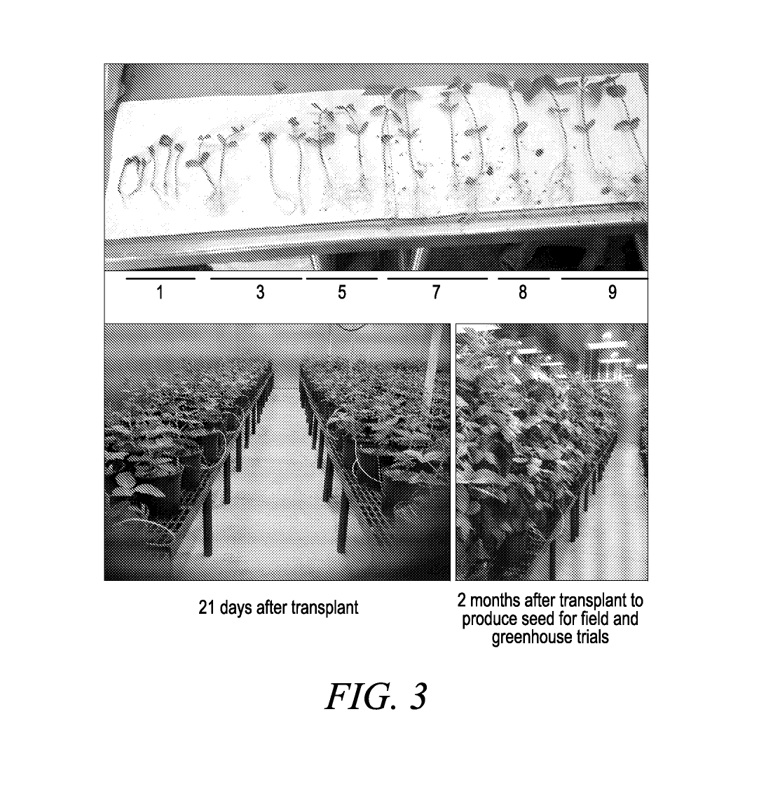

To assay all 1000 seeds, each of 17 pots (FIG. 2b) were filled with ˜250 g of course vermiculite and leveled. 60 random seeds from the synthetic bulk were then placed in a single level layer within each of 17 pots except for 1 pot which had only 40 seeds. Another 150 g of course vermiculite was then added and leveled to cover the seeds in each of the small pots. The pots were then placed in the upper tray of a MegaFarm hydroponic system as described above. The nutrient solution in the reservoir of each MegaFarm unit was made by adding 29.5 g of Peters Professional Hydroponic Special (N-P-K=5-11-26) and 19.5 grams of calcium nitrate to 30 liters of RO water (8 gallons). The nutrient solution was then supplemented to a final concentration of 0.35 PPM rimsulfuron and 22 PPM glyphosate from the herbicide stock solutions described previously. In this experiment, the hydroponic system was placed in a growth chamber providing 14 hour days at 29 degrees C. and 10 hour nights at 24 degrees C. and light quality typically used to promote vigorous plant growth. Irrigation of the pots in the upper tray with nutrient+herbicide solution was set to run for 15 minutes once every 8 hours. After 8 days of growth under the above conditions, plants were rescued from the hydroponic culture system and examined for differential response to the selective conditions. Each plant was first given a visual score of 1 (worst) through 9 (best) based on its ability to form roots and leaves under selective conditions (FIG. 3). Plants with a rating of 8 or 9 were considered diagnostic of all 3 HR genes based on pilot studies. The main difference being that plants rated 9 had slightly better development of the first trifoliate leaves than plants rated as 8.

In a practical plant breeding program, only the best-looking plants would be selected for advancement. These would be the plants most likely to contain all desired HR genes in addition to any unknown modifier genes that enhance positive response (hormesis) to the selective herbicides. However, to demonstrate the effectiveness of the current invention, all plants that emerged were given at least a visual score (FIG. 3). Plants with a rating of 8 or 9 were suspected of being homozygous for all 3 HR genes based on observation of known controls in previous pilot studies. All of these plants were therefore genotyped with genetic markers for Als1, Als2, and RR to confirm that they were indeed homozygous at the major HR loci. To further differentiate between plants that appeared to be fixed for all 3 HR genes (8 or 9 rating), fresh weight was also measured prior to transplanting into herbicide free growth chamber conditions. A sample of plants with visual ratings of 1 thru 7 were also weighed, transplanted, and genotyped to determine the rate of false negatives—i.e. plants fixed at the 3 HR loci but visually injured. MAS genotypes were also used to determine if a visual rating of 8 and/or 9 was sufficient to prevent false positives—i.e. healthy-looking plants that were not fixed at all 3 HR loci.

Although seed of the 4 lines in Table 1 were mixed together before screening, MAS genotype was used to determine what percentage of the plants within each of the visual score classes (FIG. 3) were fixed for Als1+Als2+RR and which plants lacked one or more of the 3 HR genes. Soybean line BC44883270 was the only component line of the synthetic bulk to be fixed for all 3 HR genes. Hence, by definition, any plants that confirmed as fixed for all 3 HR genes via MAS must have been members of line BC44883270. However, BC44883270 was also known to be heterogeneous (segregating) at many other non-HR loci. This genome-wide segregation was a consequence of the fact that BC44883270 was derived from a single F2 plant from a cross between an elite line 93Y92 (fixed for RR) and an experimental line W4-4 (fixed for Als1+Als2). In addition to their HR loci, 93Y92 and W4-4 are also very different from each other at many other loci throughout the genome. Hence, any visually obvious and/or measurable (e.g. weight) differences among plants of BC44883270 in response to the 2 herbicides would indicate that segregation of unknown genetic ‘modifier’ genes from either of its parents could also be affecting the whole-plant herbicide response to the selective herbicides.

### Results

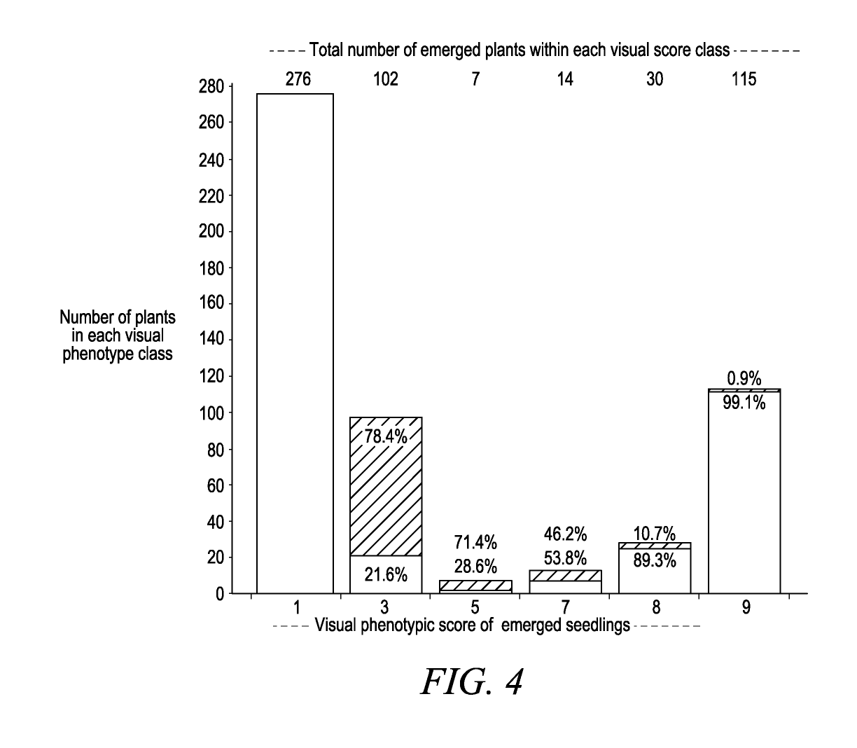

After 8 days under continuous exposure to the two selective herbicides, the full range of visual phenotypes observed in pilot studies were also observed among plants of the synthetic bulk. Plants with a rating of 3 or greater were then genotyped via MAS to determine their genotype at the Als1, Als2, and RR loci. FIG. 4 indicates both the number of plants with each of the visual scores and the percentage of plants (with a rating of 3 or greater) that independently confirmed as being fixed for Als1+Als2+RR via molecular marker genotype.

One of the most important observations was the extremely high precision of the hydroponic assay to prevent false positives—i.e. 99.1% (114 out 115) of the plants with a visual rating of 9 proved to be fixed for Als1+Als2+RR (FIG. 4). Because the components of the synthetic bulk were known, the plants fixed for Als1+Als2+RR must be members of line BC44883270 (see Table 1). Only 1 plant out of the 115 rated visually as 9 proved to be fixed for Als1+RR only (i.e. lacked Als2) via marker confirmation. This Als1+RR plant must be a member of line XB41T13 since it was the only line in the synthetic bulk of that genotype. Hence, if only plants of visual rating 9 were selected in a plant breeding program, the whole plant assay would be 99% accurate at preventing false positives. If plants with a visual rating of 8 or 9 were selected, the screen would still have been 97% accurate at preventing false positives—i.e. 141 out 145 plants confirmed as fixed for Als1+Als2+RR. This is an extremely relevant finding since the hydroponic assay could effectively eliminate most of the herbicide susceptible plants from a typical breeding population—i.e. MAS would only be necessary to confirm the homozygous state of the relatively few healthy plants. If MAS resources are limiting, progeny of the selected plants could be screened again hydroponically to expose the 1 to 3% that were false positives in the first hydroponic screen. Given the extremely low cost and relatively fast speed of the hydroponic assay versus a MAS assay, MAS confirmation would only be necessary in cases where the genotype must be confirmed immediately.

Only 58% (145 of 250) of the seeds known to be fixed for all 3 HR genes developed into healthy-looking plants (visual rating of 8 or 9) under selective herbicide conditions (FIG. 4). These 145 plants are members of line BC44883270 which was previously determined to be fixed for Als1+Als2+RR but heterogeneous (segregating) at other genomic loci. This heterogeneity is a consequence of BC44883270 being derived from a single F2 plant from a cross of 2 parents that are polymorphic at many genome-wide loci. This prior information and subsequent observation demonstrates that the hydroponic screen is not only screening for plants fixed at multiple HR loci, but is also further differentiating among said plants for segregating genetic modifiers that further enhance hormesis response. If other genetic factors were not involved in whole plant response, the wide range of phenotypes observed among plants of BC44883270 in response would not have been expected. The wide range of phenotypes displayed by individual plants of elite line XB41T13 (fixed for Als1+RR) may also be a consequence of residual heterogeneity for modifiers of HR genes. Although elite lines are typically fixed at 95% of genome-wide loci, residual segregation at even a few genetic loci can have significant effects on plant-to-plant responses.

The wide variety of phenotypic scoring among plants of Als1+Als2+RR genotype demonstrates the complex interactions of the herbicide tolerance genes with the other genes in the plants genome. Differences in these interactions may account for differences in the hormesis response among plants of a common herbicide tolerance genotype, and provide a basis to select plants in a breeding program on the basis of hormesis response according to the method of the invention, and not just genotype.

A fast, inexpensive, and accurate whole plant assay in the early generations (e.g. prior to the first yield trials) of a plant breeding cycle can dramatically improve the overall efficiency and realized genetic gain at the end of each breeding cycle. Regardless of which genetic or epigenetic factors modify the expression of HR genes, it is highly desirable to quickly eliminate all plants except those that exhibit the most vigorous growth in the presence of selective agents. This is especially true for response to selective herbicides that the plant will systematically encounter in commercial production for the purpose of controlling weeds. Since herbicides are typically applied early in the growing season, selection for improved efficacy and hormesis at the seedling stage may be especially effective to establish a healthy crop under commercial production conditions. Hence the current invention can be used to breed plants with increased productivity resulting from both weed control and maximum hormesis in response to herbicide application.

### Example 2

This example demonstrates hormesis enhancement under field conditions using combinations of herbicide resistant traits. Table 2 describes the soy lines used and whether ALS1, ALS2, or both herbicide resistant genes were present in each.

TABLE 2
Lines used in Example 2

| Line | Identifier | Genotype at 2 ALS Loci |
|---|---|---|
| 1 | BC44883342 | ALS1 |
| 2 | BC44883284 | ALS1 |
| 3 | BC44883300 | ALS2 |
| 4 | BC44883269 | ALS2 |
| 5 | BC44883336 | ALS1 + ALS2 |
| 6 | BC44883270 | ALS1 + ALS2 |
| 7 | 93M94 | ALS1 |
| | | (elite line with STS ® trait) |
| 8 | 94Y02 | ALS1 |
| | | (elite line with STS ® trait) |

Each of the lines in Table 2 was grown at a density of 150,000 seeds/acre (˜8 seeds per foot of row) in two 30 inch rows. Each line was divided into four groups, and each group of each line was subjected to one of the four herbicide treatments listed in Table 3.

TABLE 3
Herbicide treatments

| Treatment | | | Product | |
|---|---|---|---|---|
| Group | Treatment Code | Rate | Rate | Timing |
| 1 | Check | None | None | N/A |
| 2 | Tribenuron 0.25X | | | |
| | DuPont ™ Express ® | 0.03 oz ai | 0.06 oz/a | V3 |
| | w/TotalSol ® | | | |
| | Non-ionic Surfactant | 0.25% v/v | 0.25% v/v | V3 |
| | Ammonium Sulfate | 8 lb/100 gal | 8 lb/100 gal | V3 |
| 3 | Tribenuron 0.50X | | | |
| | DuPont ™ Express ® | 0.06 oz ai | 0.125 oz/a | V3 |
| | w/TotalSol ® | | | |
| | Non-ionic Surfactant | 0.25% v/v | 0.25% v/v | V3 |
| | Ammonium Sulfate | 8 lb/100 gal | 8 lb/100 gal | V3 |
| 4 | Tribenuron 1.0X | | | |
| | DuPont ™ Express ® | 0.125 oz ai | 0.25 oz/a | V3 |
| | w/TotalSol ® | | | |
| | Non-ionic Surfactant | 0.25% v/v | 0.25% v/v | V3 |
| | Ammonium Sulfate | 8 lb/100 gal | 8 lb/100 gal | V3 |

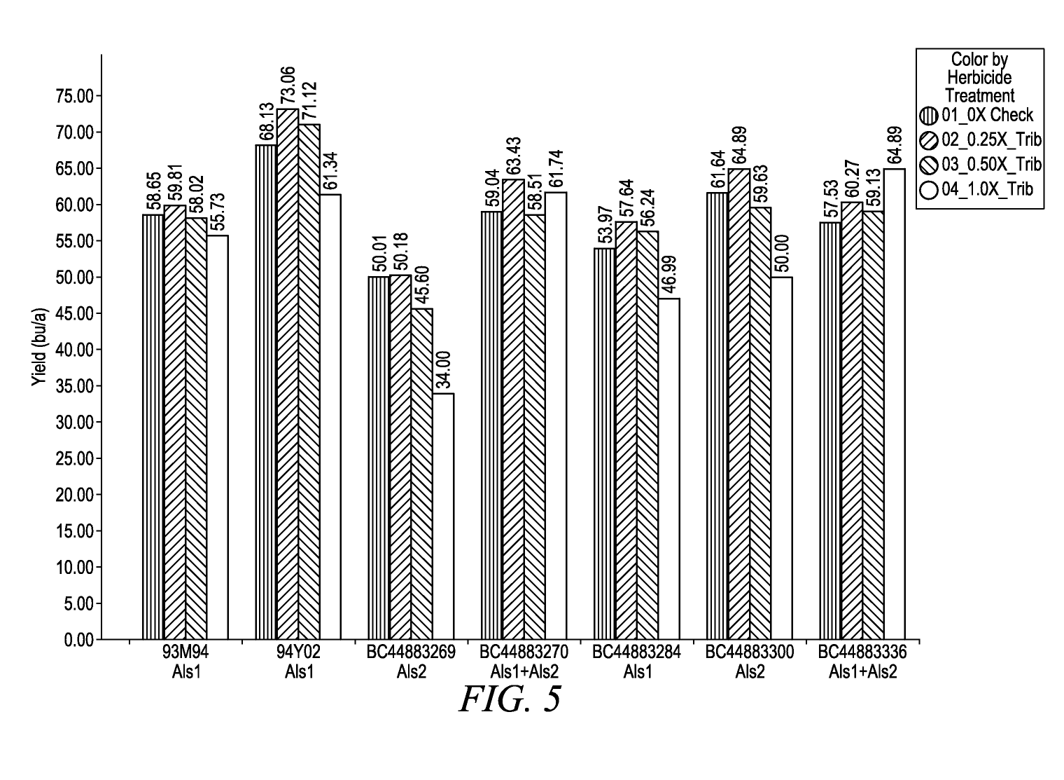

Group 1 of each line was the control group that received no herbicide treatment. Groups 2, 3, and 4 of each line were treated with different concentrations of herbicide corresponding to approximately ¼ strength, ½ strength, and full strength (needed for weed control) respectively of the recommended concentrations of the herbicide DuPont™ Express® w/TotalSol®, which includes Tribenuron, a sulfonylurea herbicide. The herbicide treatment spray solution was prepared according to the label instructions with a non-ionic surfactant and an adjuvant (ammonium sulfate). The herbicide was applied to Groups 2, 3, and 4 during the V3 growth phase, the plants were grown to maturity, and yield measurements were made. Each genotype×herbicide treatment group was replicated six times. The yield data is summarized in FIG. 5.

In FIG. 5, the y-axis represents the yield data for each line in bushels/acre. Hormesis effects are shown in all lines as demonstrated by the relative yield increase in the ¼ concentration herbicide application (blue bars) relative to the control (red bars). In both cases where both the Als1 and Als2 genes (BC44883270 and BC44883336) were present, positive hormesis effects were observed at full strength herbicide concentration (yellow bars) relative to the control (red bars). In fact, for line BC44883336, full herbicide concentration gave the best yield, a nearly 13% improvement over the control. In line BC44883270, however, the yield improvement was only about 4.5%. This example provides another demonstration of the method of the invention, in which BC44883336 would be selected in a breeding program over BC44883270 based on superior hormesis effects rather than just herbicide tolerance genotype.

Positive hormesis was outweighed by deleterious effects of the herbicides at full concentration in all of the other lines. This effect was most profound in the lines with only the Als2 gene (BC44883269 and BC44883300), with yield decreasing by about 32% and 19% respectively.

### Example 3

This example tests whether the Als1 and/or Als2 genes confer pleiotropic and/or hormesis effects in response to cold temperature germination vigor. Seeds of the varieties of soybeans listed in Table 4 were planted 1 inch deep into 800-ml Tri-Pour beakers filled with a 50/50 mixture of Matapeake soil and sand.

TABLE 4
Soy Lines Used in Example 3

| Identifier | Genotype (homozygous) |
|---|---|
| BC44883289 | Wild type (no Als1 or Als2) (control) |
| BC44883286 | Als1 |
| BC44883311 | Als2 |
| BC44883304 | Als1 and Als2 |

The pots were placed in temperature controlled root zone boxes to maintain the soil temperature at either 10° C. or at 20° C. The root zone boxes were kept in a growth room set with a 16 hour photoperiod. Five seeds were planted into each Tri-Pour beaker. There were seven replications of each soybean variety placed in each root zone chamber. There were two root zone chambers set at 10° C. and two root zone chambers set at 20° C. Each variety therefore had a total of 70 seeds exposed to each soil temperature. The Tri-Pour beakers were carefully watered as necessary to allow the soybean seeds to germinate and the resulting soybean plants to grow. Soybean germination counts were recorded for each Tri-Pour beaker on a daily basis until no more soybeans germinated. A soybean plant was considered successfully germinated when the unifoliate leaves no longer touched the cotyledons. Daily results were analyzed to determine rates of germination.

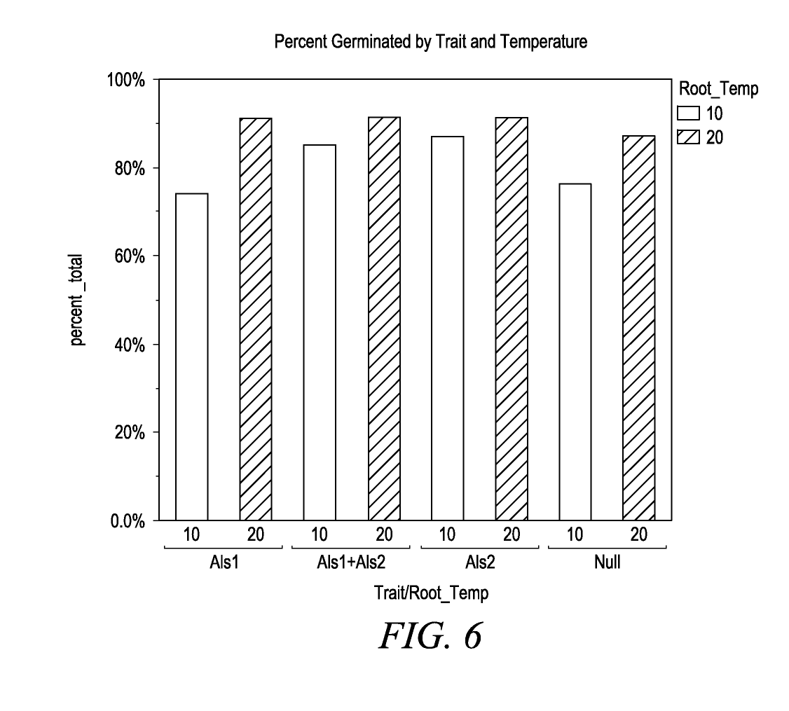

Results of the raw daily counts and average germination by day are recorded in Table 5 and displayed graphically in FIG. 6.

TABLE 5
Cold Temperature Germination

| | Root | Total | Percent |
|---|---|---|---|
| Genotype | Temperature | Germinated | Germinated |
| Als1 | 10 | 104 | 74.3% |
| Als1 | 20 | 128 | 91.4% |
| Als1 + Als2 | 10 | 119 | 85% |
| Als1 + Als2 | 20 | 128 | 91.4% |
| Als2 | 10 | 122 | 87.1% |
| Als2 | 20 | 128 | 91.4% |
| NULL (Control) | 10 | 107 | 76.4% |
| NULL (Control) | 20 | 122 | 87.1% |

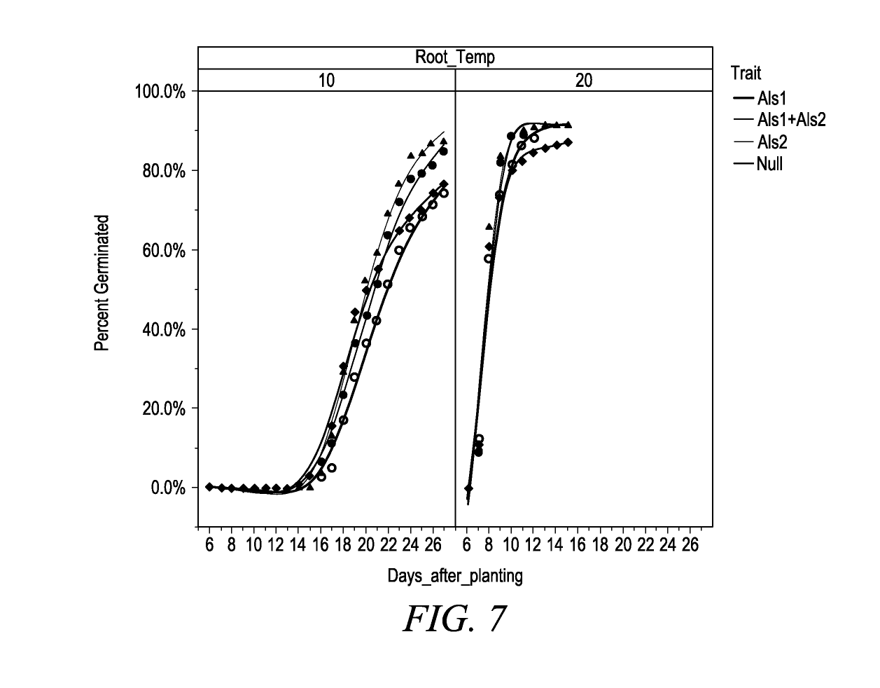

All 4 varieties had similar rates of germination in the 20° C. root zone boxes. As shown in FIG. 7, The varieties BC44883311 (Als2) and the BC44883304 (Als1+Als2) germinated sooner than the BC44883289 (null control) in the 10° C. root zone boxes, and even showed slight improvement at 20° C. The data suggests a positive correlation between genetics for ALS tolerance and better seedling vigor under colder environments. This is an unexpected pleiotropic effect of the sulfonylurea tolerance traits.

### Example 4

Another field trial similar to Example 2 was conducted with Als1+Als2 soybean lines that were more extensively backcrossed into commercial high yielding “elite” lines, referred to here as lines #8 and #9. Both elite lines already contained the RR gene and this HR trait was also maintained during backcrossing. In Example 2, the Als1+Als2 lines used (BC44883336 and BC44883270) were derived from single F2 plants of crosses W4-4×93Y82 and W4-4×93Y92. Although said lines were confirmed as homozygous HR at the Als1+Als2+RR loci, the individual F2 plant selections gave rise to lines that were segregating (heterogeneous) at many other loci throughout the genome. Hence, the lines BC44883336 and BC44883270 used in Example 2 can be described as “BC0F2-derived lines” or simply “BC0F2 lines”.

Although the term “line” implies genetic purity for a certain trait or combination of traits, inbred lines can be very heterogeneous at other genetic loci depending on the genetic differences between their parents and which generation (F2, F3, F4, etc.) a single plant was selected for subsequent bulking of seed to comprise the line. Such lines are often referred to as “heterogeneous inbred lines” (HILs) to indicate that the line is not a “pure line” or a “true-breeding line”. In other words, unless selection for a given allele is imposed, the loci that were heterozygous in the original single plant selection (e.g. a BC0F2 plant) gradually separate into a mixture of homozygous yet heterogeneous plants (e.g. 50% AA+0% Aa+50% aa).

A single “yield” measurement in an agronomic field trial is typically the weight of seed threshed from hundreds of plants that comprise the field “plot” i.e. “experimental unit”—as opposed to the seed yield of a single plant given unlimited space. This is done to mimic the actual plant population density that farmers use to maximize “yield per acre” (what they get paid for) as opposed to “yield per plant”. Hence, when measuring the relative yield of HILs in field trials, one is actually measuring the AVERAGE yield of a mixture of plants that could be quite different in terms of their genetic potential for hormesis response. So if any of the segregating loci in the HIL affect hormesis response, positive responses from plants with “hormesis-favorable” alleles or haplotypes could be masked by their admixture with plants with “hormesis unfavorable” alleles or haplotypes.

Given the above logic, further inbreeding and purification of several different Als1+Als2+RR lines was done to determine if differential hormesis responses could be detected among lines that had the same HR trait(s). This would imply that genetic background differences other than the HR genes could be affecting the hormesis response. If so, active breeding and selection for genomes that respond favorably to herbicides or other crop protection chemicals could significantly improve crop yields. If farmers are already using these chemicals for pest control, the increased crop yields could be achieved with little to no change in their current production system.

The additional backcrossing of the Als1+Als2 genes from W4-4 to the BC3 generation (4 doses of the elite parent) resulted in lines referred to as 93Y82BC3 and 93Y92BC3 respectively (Table 6). The BC3 lines are nearly isogenic with their respective elite recurrent parent—but with the addition of the Als1+Als2 genes via marker assisted selection. The BC3 lines are also more inbred than the BC0 lines used in Example 2 and therefore more homozygous and homogeneous (i.e. “pure” or “true-breeding”) throughout the entire genome in addition to purity at the major HR loci (RR, Als1, and Als2). The purified BC3 lines could then be used to test the hormesis response of pure but different genetic backgrounds (i.e. the 93Y82 vs. 93Y92 backgrounds).

TABLE 6
Soybean lines used in Example 4 and their corresponding HR genes

| | | Genotype for SU | Genotype for glyphosate |
|---|---|---|---|
| Line | Line name | resistance | resistance |
| 1 | 92Y82BC3 | Als1 + Als2 | RR |
| 2 | 92Y92BC3 | Als1 + Als2 | RR |
| 3 | 93M94 | Als1 | RR |
| 4 | 94Y02 | Als1 | RR |

In addition to the two BC3 lines containing Als1+Als2+RR, two other elite lines 93M94 and 94Y02 that contained Als1+RR but lacked the Als2 gene were also included (Table 6). Unlike Example 2, all lines in Example 4 contained the RR trait (in addition to Als1 or Als1+Als2) for several reasons. First of all, ˜90% of commercial soybean varieties are glyphosate resistant (via the RR or RR2Y trait) and secondly, glyphosate treatment is almost always used as at least one component of chemical weed control in commercial soybean production. Other herbicides combined with glyphosate (including SU's) are typically sprayed before, during, or after glyphosate treatment in order to control glyphosate resistant weeds and/or to provide additional weed control through residual activity in the soil. Hence, glyphosate was used as the most commercially-relevant control treatment applied to the entire field. Glyphosate application also helped to maintain weed-free conditions throughout the field trials such that yield responses were not affected by differential weed pressure among plots.

The field trial was conducted as a 7×4 factorial experiment (7 herbicide regimes×4 genotypes) in a split block design with main blocks as herbicide treatments. The trial was conducted at 3 different environments (separate field locations) in Iowa during the summer of 2014. Herbicide control treatment #1 (no sulfonylurea) was replicated 12 times within each of the 3 environments (36 reps total). Herbicide treatments 2 through 7 (Table 7) were replicated 6 times at each of the 3 environments (18 reps total). The additional replication of control treatment #1 was done to increase precision of the treatment mean to which all other treatments (#2 through 7) would be compared.

It is important to note that both tribenuron and rimsulfuron are more toxic to wild type soybeans than other SU's that could also be tested. But treatment with these specific SU's and application rates was intended to push the limits of SU tolerance so that any response differences between lines could be exposed. The 4 genotypes (Table 6) were randomized within each of the main blocks to facilitate post-emergence application of the various herbicide treatments (Table 7). Each experimental unit was a 2-row soybean plot 15 feet long with 30 inch row spacing and a planting density of 8 seeds per foot of row. Planting was done in mid-May and harvest was done in early October at all 3 environments in 2014.

Several weeks after planting, the entire field was sprayed post-emergence at the V2 (2-leaf) stage with Roundup PowerMax® at 44 oz/acre (30 oz/acre ai glyphosate) with the addition of ammonium sulfate at a rate of 8 lb per 100 gallons as according to label. Several days later at the V3 (3-leaf) stage, herbicide treatments 2 through 7 (Table 7) were applied according to label directions including the addition of ammonium sulfate at a rate of 8 lb per 100 gallons of spray solution. Randomly embedded control plots of a given soybean genotype facilitated side-by-side visual estimates of herbicide injury. Plots were given visual injury ratings at 14 days after herbicide treatment (14 DAT). Visual injury estimates were assigned to reflect the crop response (a combination of reduced vigor and/or chlorosis) of sprayed rows in relation to the randomly embedded control plots. Injury scores were based on a 0-100% scale, with 0 indicating no crop response and 100 indicating all plants killed.

TABLE 7
Herbicide treatments in Example 4-2014 field hormesis trial

| | | | Active | Formulated |
|---|---|---|---|---|
| Trt | Treatment | Herbicide | ingredient | product |
| # | description | product name | (oz/acre) | (oz/acre) |
| 1 | Control | no sulfonylurea | 0 | 0 |
| | | applied | | |
| 2 | Tribenuron 0.5X | Express ® w/ | 0.125 oz/a | 0.25 oz/a |
| | | TotalSol ® | | |
| 3 | Tribenuron 1X | Express ® w/ | 0.25 oz/a | 0.50 oz/a |
| | | TotalSol ® | | |
| 4 | Tribenuron 2X | Express ® w/ | 0.50 oz/a | 1.00 oz/a |
| | | TotalSol ® | | |
| 5 | Rimsulfuron 0.5X | Resolve ® DF | 0.25 oz/a | 1.00 oz/a |
| 6 | Rimsulfuron 1X | Resolve ® DF | 0.50 oz/a | 2.00 oz/a |
| 7 | Rimsulfuron 2X | Resolve ® DF | 1.00 oz/a | 4.00 oz/a |

At maturity, the seed from each plot was harvested with weight and moisture recorded. Weights were then adjusted to 13% moisture content and reported in units of bushels per acre (bu/a). Yields were also converted to % of control on a “per line” and “per environment” basis. For example, the average yield of SU-treated 93Y92BC3 plots were compared to the average yield of 93Y92BC3 control plots within a given environment and then averaged across environments. The yield data are summarized in Table 8.

TABLE 8
Visual injury and yield response of various soybean lines to herbicide application

| | | | | Visual | | Yield | Yield as | |
|---|---|---|---|---|---|---|---|---|
| Soybean | HR | Herbicide | N | injury | Yield | SEM | % of | Yield |
| line | genotype | Treatment | (# obs) | 14 DAT | (bu/a) | (bu/a) | control | effect |
| 93M94 | RR + Als1 | 1: control | 36 | 0 | 48.2 | 1.2 | 100 | control |
| 93M94 | RR + Als1 | 2: tribenuron 0.5x | 18 | 10 | 47.5 | 1.5 | 98 | neutral |
| 93M94 | RR + Als1 | 3: tribenuron 1x | 18 | 18 | 48.9 | 1.5 | 101 | neutral |
| 93M94 | RR + Als1 | 4: tribenuron 2x | 18 | 39 | 41.9 | 1.5 | 87 | negative |
| 93M94 | RR + Als1 | 5: rimsulfuron 0.5x | 18 | 72 | 36.7 | 1.5 | 76 | negative |
| 93M94 | RR + Als1 | 6: rimsulfuron 1x | 18 | 86 | 24.7 | 1.5 | 51 | negative |
| 93M94 | RR + Als1 | 7: rimsulfuron 2x | 18 | 87 | 19.6 | 1.5 | 41 | negative |
| 94Y02 | RR + Als1 | 1: control | 36 | 0 | 59.2 | 1.2 | 100 | control |
| 94Y02 | RR + Als1 | 2: tribenuron 0.5x | 18 | 9 | 58.8 | 1.5 | 99 | neutral |
| 94Y02 | RR + Als1 | 3: tribenuron 1x | 18 | 19 | 58.2 | 1.5 | 98 | neutral |
| 94Y02 | RR + Als1 | 4: tribenuron 2x | 18 | 39 | 48.6 | 1.5 | 82 | negative |
| 94Y02 | RR + Als1 | 5: rimsulfuron 0.5x | 18 | 78 | 31.7 | 1.5 | 54 | negative |
| 94Y02 | RR + Als1 | 6: rimsulfuron 1x | 18 | 87 | 19.5 | 1.5 | 33 | negative |
| 94Y02 | RR + Als1 | 7: rimsulfuron 2x | 18 | 81 | 12.2 | 1.5 | 21 | negative |
| 93Y82BC3 | RR + Als1 + Als2 | 1: control | 36 | 0 | 60.5 | 1.2 | 100 | control |
| 93Y82BC3 | RR + Als1 + Als2 | 2: tribenuron 0.5x | 18 | 3 | 61.1 | 1.5 | 101 | neutral |
| 93Y82BC3 | RR + Als1 + Als2 | 3: tribenuron 1x | 18 | 6 | 61.3 | 1.5 | 101 | neutral |
| 93Y82BC3 | RR + Als1 + Als2 | 4: tribenuron 2x | 18 | 16 | 61.4 | 1.5 | 102 | neutral |
| 93Y82BC3 | RR + Als1 + Als2 | 5: rimsulfuron 0.5x | 18 | 54 | 60.1 | 1.5 | 99 | neutral |
| 93Y82BC3 | RR + Als1 + Als2 | 6: rimsulfuron 1x | 18 | 62 | 56.2 | 1.5 | 93 | negative |
| 93Y82BC3 | RR + Als1 + Als2 | 7: rimsulfuron 2x | 18 | 69 | 48.7 | 1.5 | 81 | negative |
| 93Y92BC3 | RR + Als1 + Als2 | 1: control | 36 | 0 | 55.8 | 1.2 | 100 | control |
| 93Y92BC3 | RR + Als1 + Als2 | 2: tribenuron 0.5x | 18 | 2 | 59.4 | 1.5 | 106 | Positive |
| 93Y92BC3 | RR + Als1 + Als2 | 3: tribenuron 1x | 18 | 5 | 58.0 | 1.5 | 104 | Positive |
| 93Y92BC3 | RR + Als1 + Als2 | 4: tribenuron 2x | 18 | 14 | 60.0 | 1.5 | 108 | Positive |
| 93Y92BC3 | RR + Als1 + Als2 | 5: rimsulfuron 0.5x | 18 | 47 | 59.8 | 1.5 | 107 | Positive |
| 93Y92BC3 | RR + Als1 + Als2 | 6: rimsulfuron 1x | 18 | 59 | 60.5 | 1.5 | 108 | Positive |
| 93Y92BC3 | RR + Als1 + Als2 | 7: rimsulfuron 2x | 18 | 66 | 49.9 | 1.5 | 89 | negative |

The 3 Iowa environments tested in 2014 were of sufficiently favorable climate to support soybean yields typical for Iowa USA (control yields of 50 to 60 bu/a). In other words, yields in these field environments were not unusually suppressed due to environmental conditions that might limit the expression of hormesis for seed yield induced by herbicide treatment.

It is important to note again that both tribenuron and rimsulfuron are much more toxic (i.e. active at lower rates) to soybeans than other SU's that could have been sampled. Treatment with these specific SU's and rates was intended to push the limits of SU tolerance so that any differential response between Als1-only and Als1+Als2 lines could be exposed. Given that 2 different lines of each HR genotype were tested (Table 8), the experiment could also detect differential responses between lines with identical HR genes but with different genetic backgrounds.

The SU tolerance of lines with Als1+Als2 was superior to the SU tolerance of lines containing Als1 only. This was evident in both visual injury scores at 14 DAT and in final seed yields (Table 8). Based on past experience with both wild type and Als1-only lines in response to a wide range of SU treatments, injury ratings of greater than 20% at 14 DAT usually result in negative yield responses at harvest. This yield versus injury response was confirmed for the Als1 lines 93M94 and 94Y02 in the current example. Both Als1-only lines had significant yield depression when 14 DAT injury ratings exceeded 20%, regardless of the SU treatment.

In contrast, lines with Als1+Als2 recovered much faster from visual injury observed at 14 DAT—regardless of the SU treatment. Although not recorded, the Als1+Als2 lines had a dramatic recovery from obvious injury by 30 days after treatment. This fast recovery between 14 and 30 DAT is clearly reflected in the final seed yields. For example, the Als1+Als2 lines could sustain up to 59% visual injury at 14 DAT without any negative impact on final seed yield. This is a very unique feature of the Als1+Als2 lines in contrast to common assumptions about the relationship between visual herbicide injury and final yield response. Although Als1+Als2 line 93Y82BC3 did not express a significantly-positive yield response to the SU treatments, it maintained yield stability (within 1 to 2% of the control treatment) even after sustaining 54% visual injury at 14 DAT (e.g. treatment 5).

In addition to no yield depression, Als1+Als2 line 93Y92BC3 displayed a positive yield response (yield hormesis) in the range of 4% to 8% versus control at all 3 treatments of tribenuron (0.5×, 1×, and 2×) and at 2 of the 3 rimsulfuron treatments (0.5× and 1×).

The present example also demonstrates that the hormesis response can be triggered at SU rates that cause little to no visible injury. This is demonstrated by herbicide treatments 2 and 3 which had an average visual injury of 6% or less on both Als1+Als2 lines. For reference purposes, injury ratings of less than 10% are within the range of experimental error on a single plot basis. So, it would be difficult for a soybean grower to even detect 6% injury at 14 DAT or to conclude that said injury was caused by herbicide application as opposed to other sources of spatial variation that are typical of field yield trials. This is also why 18 to 36 replications of yield data are typically required to detect true yield differences that are greater than 4 to 5% of relevant controls.

Given the wide variety of ALS-inhibiting herbicides (including SU's) that are commercially available, it is reasonable to expect that other ALS-inhibiting herbicides can trigger a positive yield response—especially with the wide safety window afforded by the Als1+Als2 genes. In addition, it is reasonable to expect a similar or even wider range of positive responses when these HR genes are available in a wider variety of genetic backgrounds. It is also expected that empirical testing of other herbicides (or other crop protection chemicals) and genotype combinations could reveal other treatments that trigger hormesis.

In conclusion, Als1+Als2 can significantly reduce SU herbicide injury, can speed recovery from herbicide injury, and can stabilize yield potential when compared to lines with Als1 alone. This example also reveals that 2 different lines (93Y82BC3 and 93Y92BC3) containing the same major HR genes can differ greatly in terms of positive yield response (hormesis) to herbicide treatment. Therefore, it is apparent that genetic background differences other than major HR genes can significantly affect the occurrence and/or magnitude of the hormesis response. Hence, active selection for genetic backgrounds that maximize the hormesis response is both possible and highly desirable for the purpose of maximizing crop yields. Given that the examples herein have barely sampled all possible combinations of HR genes, genetic backgrounds, and herbicide treatments, it is likely that other genotype+herbicide combinations could also be leveraged to maximize crop yields. It is also conceivable that other types of crop protection chemicals (insecticides, nematicides, fungicides, plant growth regulators, etc.) could trigger differential hormesis responses within specific genetic backgrounds of any crop.

### Example 5

### Effects of Glyphosate Treatment on Growth of Glyphosate Tolerant Make Inbred Lines

Seeds from four glyphosate tolerant maize inbred lines (PH1BVW2, PH1PMB1, PHSZB1 and PH19081) were washed in 0.615% NaClO solution for 5 minutes and rinsed with deionized water. They were germinated for one week and then transferred into individual 10″ Deepot tubes (1 seedling/tube), either with foam plugs to suspend the plants in the tubes (Experiments 1 &2) or filled with Turface (Experiments 3 & 4). These tubes were placed into hydroponic growing tanks (100 tubes/tank) with a modified Hoagland's media. At the end of the 2nd week after planting, plants from selected tanks were sprayed with glyphosate solutions equivalent to 1× or 2× of the recommended dosage (1× dosage: 1 quart of liquid Round-Up WeatherMax® per acre, or 21.75 ul/ft2). Separately, three inbred lines without the glyphosate tolerance trait (PH1BVW2, PH1PMB1, and PHSZB1) were treated with the same dosages of glyphosate to confirm the efficacy of the herbicide. The inbred lines without the glyphosate tolerance trait were killed by both glyphosate treatments, as expected. Four weeks after germination, plants were harvested and shoot fresh weight was recorded. Table 8 lists the results.

TABLE 8
Differential Hormesis response among RR ®
maize lines treated with glyphosate

| | | | Number of | Mean fresh |
|---|---|---|---|---|
| Experiment | Genotype | Treatment | plants | weight (g) |
| 1 | PH1BVW2 | Control | 25 | 8.7 |
| 1 | PH1BVW2 | 1x-RoundUp | 25 | 11.6 |
| 1 | PH1BVW2 | 2x-RoundUp | 25 | 11.8 |
| 1 | PH1PMB1 | Control | 25 | 54.2 |
| 1 | PH1PMB1 | 1x-RoundUp | 25 | 47.2 |
| 1 | PH1PMB1 | 2x-RoundUp | 25 | 53.6 |
| 1 | PHSZB1 | Control | 25 | 43.7 |
| 1 | PHSZB1 | 1x-RoundUp | 25 | 39.0 |
| 1 | PHSZB1 | 2x-RoundUp | 25 | 42.5 |
| 1 | PH19081 | Control | 25 | 9.2 |
| 1 | PH19081 | 1x-RoundUp | 25 | 17.6 |
| 1 | PH19081 | 2x-RoundUp | 25 | 12.4 |
| 2 | PH1BVW2 | Control | 50 | 13.6 |
| 2 | PH1BVW2 | 1x-RoundUp | 25 | 7.9 |
| 2 | PH1BVW2 | 2x-RoundUp | 25 | 16.9 |
| 2 | PH1PMB1 | Control | 50 | 48.8 |
| 2 | PH1PMB1 | 1x-RoundUp | 25 | 54.7 |
| 2 | PH1PMB1 | 2x-RoundUp | 25 | 59.7 |
| 2 | PHSZB1 | Control | 50 | 38.8 |
| 2 | PHSZB1 | 1x-RoundUp | 25 | 35.3 |
| 2 | PHSZB1 | 2x-RoundUp | 25 | 39.0 |
| 2 | PH19081 | Control | 50 | 8.6 |
| 2 | PH19081 | 1x-RoundUp | 25 | 9.3 |
| 2 | PH19081 | 2x-RoundUp | 25 | 10.1 |
| 3 | PH1BVW2 | Control | 25 | 42.7 |
| 3 | PH1BVW2 | 1x-RoundUp | 25 | 47.4 |
| 3 | PH1BVW2 | 2x-RoundUp | 25 | 53.5 |
| 3 | PH1PMB1 | Control | 25 | 90.5 |
| 3 | PH1PMB1 | 1x-RoundUp | 25 | 74.5 |
| 3 | PH1PMB1 | 2x-RoundUp | 25 | 86.7 |
| 3 | PHSZB1 | Control | 25 | 78.9 |
| 3 | PHSZB1 | 1x-RoundUp | 25 | 67.4 |
| 3 | PHSZB1 | 2x-RoundUp | 25 | 65.5 |
| 3 | PH19081 | Control | 25 | 15.1 |
| 3 | PH19081 | 1x-RoundUp | 25 | 18.0 |
| 3 | PH19081 | 2x-RoundUp | 25 | 16.3 |
| 4 | PH1BVW2 | Control | 100 | 29.1 |
| 4 | PH1BVW2 | 1x-RoundUp | 100 | 27.3 |
| 4 | PH1BVW2 | 2x-RoundUp | 100 | 28.9 |
| 4 | PH19081 | Control | 100 | 16.5 |
| 4 | PH19081 | 1x-RoundUp | 100 | 17.8 |
| 4 | PH19081 | 2x-RoundUp | 100 | 17.2 |

In this example, it was observed that some of the lines (PH1BVW2 and PH19081) demonstrate a positive hormesis effect upon application of herbicide while the others did not. This example demonstrates several points in addition to: 1) hormesis can be expressed as an increase in seedling fresh weight or vigor, 2) that breeding selections to maximize hormesis effects in maize can be made to improve crop vigor and 3) species besides soybean also exhibit hormesis.

### Example 6

### Hormesis Effects with Seed Applied Components

Seeds are produced that have tolerance to one or more herbicides, for example, tolerance to glyphosate and rimsulfuron. The seeds are selected from plants that demonstrated a positive hormesis response when exposed to the herbicides to which they are tolerant. The seeds are coated with one or more herbicides to which they are tolerant, in this example glyphosate and/or rimsulfuron. In an embodiment, the herbicide concentration is at a non-lethal level to a seed or a plant that does not exhibit substantial tolerance to that herbicide. In an embodiment, the herbicide concentration is at a level that is adequate to induce hormesis in a seed or a plant that exhibits substantial tolerance to that herbicide. The coating may include at least one biodegradable polymer to assist in adhesion and durability of the coating. The coating may also include, optionally an insecticide, a fungicide, a biological organism and/or a colorant. The coated seeds are planted, and an agronomic characteristic such as for example, increased vigor, germination, standability, plant health, fresh plant weight, and yield are expected relative to uncoated seeds of the same variety. Seeds treated with a seed treatment can also have one or more transgenic traits including for example, insect tolerance, disease resistance, drought tolerance, increased nutrient or nitrogen use efficiency and a combination thereof. Hormesis can also be accomplished by providing a seed treatment that includes exogenous application of nucleotides (e.g., single or double stranded DNA or RNA) targeting one or more endogenous genes of a plant species or pest species. Glyphosate tolerance is due to the expression of a glyphosate insensitive EPSP synthase (EPSPS) or a glyphosate detoxification enzyme such as glyphosate acetyl transferase (GAT). While the foregoing examples were in soy and maize, on the basis of these examples and the disclosure, those of ordinary skill in the art would understand that the same types of effects would be expected in other plant species including canola, sunflower, rice, alfalfa, sorghum, and wheat, or any other plant species. Likewise, based on these results, those in the agricultural arts would expect that it also possible to select for improved hormesis in response to other types of chemicals besides herbicides—including insecticides, fungicides, and nematicides. Since these chemicals are not specifically designed to kill plants, positive hormesis responses may occur at normal use rates without the need for major genes conditioning specific resistance to said chemicals.

1. A method of selecting plant lines with a hormesis response comprising:
a. growing multiple plant lines;
b. applying a stress in the form of a herbicide;
c. observing an increased yield response (in bushels per acre) to the stress compared to corresponding control plants with the same genetic profile without having the herbicide applied in one or more of the plant lines indicating a hormesis effect; and
d. selecting the plant lines with the strongest increased yield responses to the stress; and
e. growing the plant lines selected for the greatest increased yield responses to the stress relative to the corresponding control plant yields.

2. (canceled)

3. The method of claim 2, wherein the plant lines have a tolerance trait to the herbicide.

4. (canceled)

5. (canceled)

6. A method of producing seed with improved vigor comprising:
a. growing a parent plant under stress conditions comprising application of an herbicide for part or all of the plant's life cycle; and
b. collecting seed from the parent plant, wherein said seed has improved vigor over seed grown from the same parental line grown to maturity without the stress condition.

7. (canceled)

8. (canceled)

9. (canceled)

10. (canceled)

11. (canceled)

12. (canceled)

13. (canceled)

14. A method for increasing crop yield comprising:
selecting multiple crop lines with resistance to an herbicide;
growing test lines and control lines corresponding to each of the test lines;
applying at least one treatment of the herbicide to each of the test lines while avoiding applying the herbicide to each of the corresponding control lines;
measuring the yield response (in bushels per acre) of the herbicide treatments in each of the lines relative to the corresponding control lines; and
selecting the test lines having an increased yield response to the herbicide treatments relative to the corresponding control lines.

15. (canceled)

16. The method of claim 14, wherein the crop is soy or corn.

17. (canceled)

18. The method of claim 14, wherein the herbicide is selected from the group consisting of glyphosate, rimsulfuron, and tribenuron.

19. The method of claim 18, wherein the crop is corn or soy.

20. The method of claim 1, wherein the plants are soy plants with glyphosate and sulfonylurea tolerance, the soy crop having transgenic glyphosate tolerance and native sulfonylurea tolerance.

21. The method of claim 14, wherein the crop is soy with glyphosate and sulfonylurea tolerance, the soy crop having transgenic glyphosate tolerance and native sulfonylurea tolerance.
